(19) United States
(12) Patent Application Publication
(10) Pub. No.: US 2008/0194817 A1
(43) Pub. Date: Aug. 14, 2008

(54) Reactive Dyestuffs, Method for the Production Thereof, and Use of the Same

(75) Inventors: Stefan Meier, Frankfurt (DE); Uwe Reiher, Hofheim (DE); Werner Hubert Russ, Florsheim-Wicker (DE)

Correspondence Address: CONNOLLY BOVE LODGE & HUTZ, LLP, P O BOX 2207, WILMINGTON, DE, 19899, US

(73) Assignee: DyStar Textilfarben GmbH & Co. Deutschland KG, Frankfurt am Main (DE)

(21) Appl. No.: 11/909,684

(22) PCT Filed: Mar. 21, 2006
(86) PCT No.: PCT/EP2006/060927

§ 371 Date: Nov. 16, 2007

(30) Foreign Application Priority Data
Mar. 26, 2005 (DE) 102005013960.4

(51) Int. Cl. C07D 251/12

(52) U.S. Cl. 544/211

(57) ABSTRACT

Dyes of the hereinbelow indicated and defined formula (1)

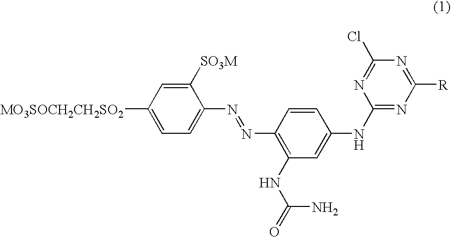

where R and M are each as defined in claim 1, process for their preparation and their use for dyeing and printing hydroxyl- and carboxamido-containing material.

This invention resides in the technical field of fiber-reactive azo dyes.

Reactive dyes useful for dyeing cellulose fibers are known and extensively described in the patent literature. However, these conventional dyes do not adequately satisfy the latest high expectations of reactive dyes. Especially the buildup performance of many reactive dyes is frequently in need of improvement. There therefore continues to be a need for new fiber-reactive dyes having improved properties. Especially the production of dyeings having a yellow hue requires reactive dyes possessing a high fastness level and a very good color buildup.

DE 4423650 describes yellow-dyeing fiber-reactive dyes which, however, do not adequately satisfy the stated criteria in that especially the color buildup of these products on cellulose fibers is still unsatisfactory.

The present inventors have now found dyes which surprisingly have a distinctly better buildup than the dyes described in DE4423650.

The present invention accordingly provides dyes of the hereinbelow indicated and defined formula (1)

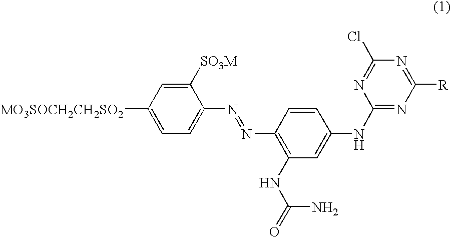

where

M is hydrogen or an alkali metal, such as sodium, potassium or lithium

R is a monoazo dye of the formulae (2a) to (2i)

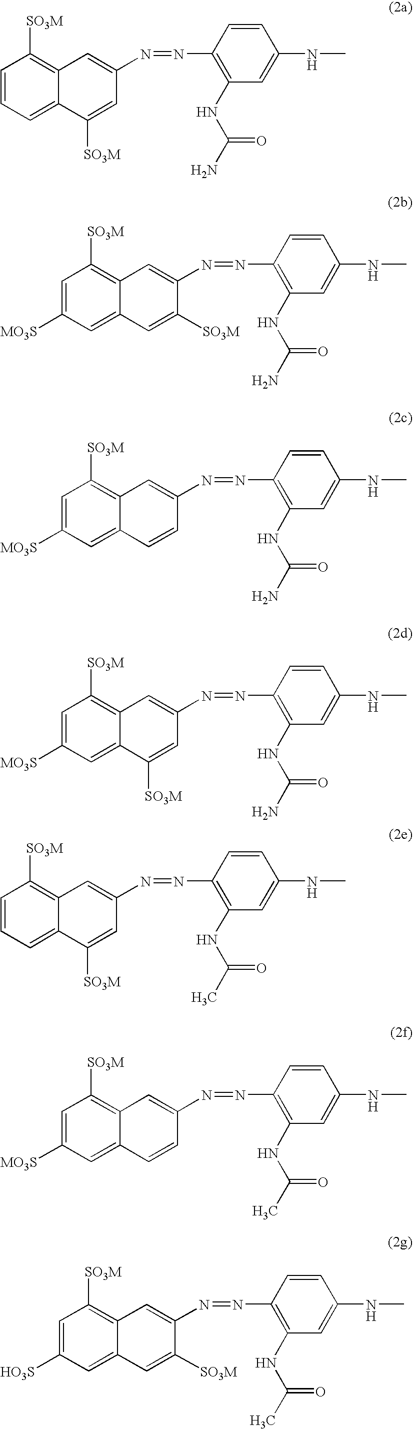

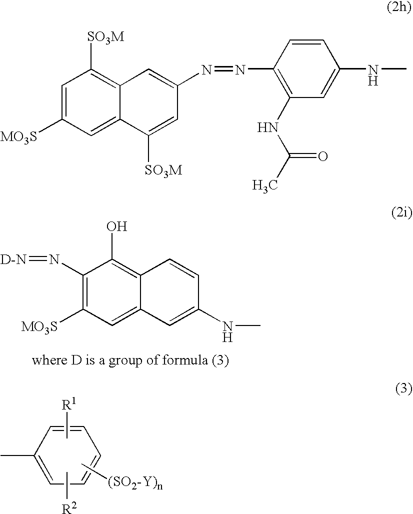

where

R¹ is hydrogen, methyl, methoxy, sulfo or chlorine,

R² has the meaning of R¹,

n is zero or 1 (this group being hydrogen in the case of n being zero) and

Y is vinyl or is ethyl substituted in the β position by a substituent which is alkali eliminable to leave a vinyl group, examples being chlorine, bromine, acetyloxy, p-tolylsulfonyloxy, thiosulfato, phosphate and especially sulfato or β-hydroxyethyl.

The dyes of the formula (1) wherein R has one of the meanings of the formulae (2a) to (2h) are prepared by the monoazo compound of the formula (4)

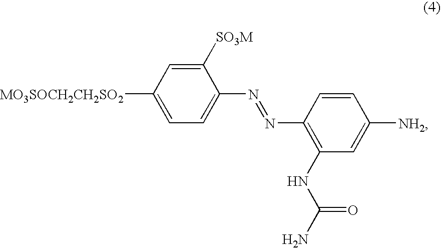

where M is as defined above, being acylated with cyanuric chloride and subsequently condensed with a monoazo dye of the formula (5)

R—H (5)

where R has the meaning identified in the formulae (2a) to (2h).

The monoazo compound of the formula (4) is known from the German patent publication DE4425222 and can be prepared similarly to the directions given therein.

The monoazo compounds of the formula (5) are extensively described in the patent literature and so are obtainable via standard methods of synthesis.

To prepare the reactive dyes of the formula (1) wherein R has the meaning of one of the formulae (2a) to (2h), the acylation of the monoazo compound of the formula (4) with cyanuric chloride and also the subsequent condensation with the monoazo compound of the formula (5) takes place in the weakly acidic to neutral range. The reaction temperature is 20-30° C. for the acylation and 60-80° C. for the condensation.

The dyes of the formula (1) wherein R has the meaning of the formula (2i) are prepared by the monoazo compound of formula (4), where M is as defined above, being acylated with cyanuric chloride and then condensed with a compound of the formula (6).

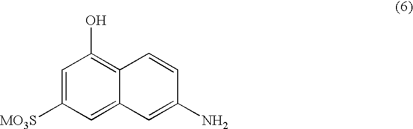

The resulting condensation product is subsequently reacted with the diazonium compound of an amine of the general formula (7)

D-NH₂ (7)

where D is as defined above.

The acylation of the monoazo compound of the formula (4) with cyanuric chloride and also the subsequent condensation with a compound of the formula (6) are carried out in the weakly acidic to neutral range. The reaction with a diazonium compound of an amine of the general formula (7) is likewise carried out in the weakly acidic to neutral range.

The dyes of the present invention possess useful application properties. They are used for dyeing or printing hydroxyl- and/or carboxamido-containing materials, for example in the form of sheetlike structures, such as paper and leather or of films, as for example of polyamide, or in the mass, as for example of polyamide and polyurethane, but in particular for these materials in fiber form Similarly, the as-synthesized solution of the dyes of the present invention can be used directly as a liquid preparation in dyeing, if appropriate after addition of a buffer substance, if appropriate also after concentrating or diluting.

The present invention thus is also directed to the use of the dyes of the present invention for dyeing or printing these materials or as the case may be to processes for dyeing or printing such materials in a conventional manner wherein the dyes are neutralized as colorants. The materials are preferably employed in the form of fiber materials, especially in the form of textile fibers, such as wovens or yarns, as in the form of hanks or wound packages.

The dye mixtures of the invention can also be used in digital printing processes, particularly in digital textile printing. For that purpose it is necessary to formulate the dye mixtures of the invention in inks. Aqueous inks for digital printing which characteristically comprise a dye of the invention are likewise provided by the present invention.

The inks of the invention contain the dyes of the invention preferably in amounts from 0.1% to 50% by weight, more preferably in amounts from 1% to 30% by weight and very preferably in amounts from 1% to 15% by weight, based on the total weight of the ink. In addition to the dyes of the invention the inks may include, where desired, further reactive dyes which are used in digital printing.

For the use of the inks of the invention in a continuous flow process electrolyte can be added to set a conductivity of 0.5 to 25 mS/m. Examples of suitable electrolyte include lithium nitrate and potassium nitrate.

The inks of the invention may include organic solvents with a total content of 1-50%, preferably of 5-30% by weight. Examples of suitable organic solvents are alcohols, such as methanol, ethanol, 1-propanol, isopropanol, 1-butanol, tert-butanol and pentyl alcohol, for example; polyhydric alcohols, such as 1,2-ethanediol, 1,2,3-propanetriol, butanediol, 1,3-butanediol, 1,4-butanediol, 1,2-propanediol, 2,3-propanediol, pentanediol, 1,4-pentanediol, 1,5-pentanediol, hexanediol, D,L-1,2-hexanediol, 1,6-hexanediol, 1,2,6-hexanetriol and 1,2-octanediol, for example; polyalkylene glycols, such as polyethylene glycol and polypropylene glycol, for example; alkylene glycols having 1 to 8 alkylene groups, such as monoethylene glycol, diethylene glycol, triethylene glycol, tetraethylene glycol, thioglycol, thiodiglycol, butyltriglycol, hexylene glycol, propylene glycol, dipropylene glycol and tripropylene glycol, for example; lower alkyl ethers of polyhydric alcohols, such as ethylene glycol, monomethyl ether, ethylene glycol monoethyl ether, ethylene glycol monobutyl ether, diethylene glycol monomethyl ether, diethylene glycol monoethyl ether, diethylene glycol monobutyl ether, diethylene glycol monohexyl ether, triethylene glycol monomethyl ether, triethylene glycol monobutyl ether, tripropylene glycol monomethyl ether, tetraethylene glycol monomethyl ether, tetraethylene glycol monobutyl ether, tetraethylene glycol dimethyl ether, propylene glycol monomethyl ether, propylene glycol monoethyl ether, propylene glycol monobutyl ether and tripropylene glycol isopropyl ether, for example; polyalkylene glycol ethers, such as polyethylene glycol monomethyl ether, polypropylene glycol glycerol ether, polyethylene glycol tridecyl ether and polyethylene glycol nonylphenyl ether, for example; amines, such as methylamine, ethylamine, triethylamine, diethylamine, dimethylamine, trimethylamine, dibutylamine, diethanolamine, triethanolamine, N-acetylethanolamine, N-formylethanolamine and ethylenediamine, for example; urea derivatives, such as urea, thiourea, N-methylurea, N,N′-epsilon-dimethylurea, ethyleneurea and 1,1,3,3-tetramethylurea, for example; amides, such as dimethylformamide, dimethylacetamide and acetamide, for example; ketones or keto alcohols, such as acetone and diacetone alcohol, for example; cyclic ethers, such as tetrahydrofuran, trimethylolethane, trimethylolpropane, 2-butoxyethanol, benzyl alcohol, 2-butoxyethanol, gamma-butyrolactone, epsilon-caprolactam, for example; additionally sulfolane, dimethylsulfolane, methylsulfolane, 2,4-dimethylsulfolane, dimethyl sulfone, butadiene sulfone, dimethyl sulfoxide, dibutyl sulfoxide, N-cyclohexylpyrrolidone, N-methyl-2-pyrrolidone, N-ethylpyrrolidone, 2-pyrrolidone, 1-(2-hydroxyethyl)-2-pyrrolidone, 1-(3-hydroxypropyl)-2-pyrrolidone, 1,3-dimethyl-2-imidazolidinone, 1,3-dimethyl-2-imidazolinone, 1,3-bismethoxymethylimidazolidine, 2-(2-methoxyethoxy)ethanol, 2-(2-ethoxyethoxy)ethanol, 2-(2-butoxyethoxy)ethanol, 2-(2-propoxyethoxy)ethanol, pyridine, piperidine, butyrolactone, trimethylpropane, 1,2-dimethoxypropane, dioxane, ethyl acetate, ethylenediaminetetraacetate, ethyl pentyl ether, 1,2-dimethoxypropane and trimethylpropane.

The inks of the invention may further include the customary additives, such as, for example, viscosity moderators to set viscosities in the range from 1.5 to 40.0 mPa·s in a temperature range from 20 to 50° C. Preferred inks have a viscosity of 1.5 to 20 mPas and particularly preferred inks a viscosity of 1.5 to 15 mPas.

Useful viscosity moderators include Theological additives, examples being the following: polyvinylcaprolactam, polyvinylpyrrolidone and their copolymers, polyether polyol, associative thickener, polyurea, polyurethane, sodium alginates, modified galactomannans, polyetherurea, polyurethane, and nonionic cellulose ethers.

As further additions the inks of the invention may include surface-active substances to set surface tensions of 20 to 65 mN/m, which are adapted where appropriate as a function of the process used (thermal or piezo technology). Useful surface-active substances include, for example, surfactants of all kinds, preferably nonionic surfactants, butyldiglycol and 1,2-hexanediol.

The inks of the invention may further include customary additions, such as substances for preventing fungal and bacterial growth, for example, in amounts of 0.01% to 1% by weight, based on the total weight of the ink.

The inks may be prepared in a conventional manner by mixing the components in water.

The inks of the invention are especially useful for use in inkjet printing processes for printing a wide variety of pretreated materials, such as silk, leather, wool, polyamide fibers and polyurethanes, and especially cellulosic fiber materials of any kind. Blend fabrics as well can be printed, examples being blends of cotton, silk or wool with polyester fibers or polyamide fibers.

In contrast to conventional textile printing, where the printing ink already contains all the fixing chemicals and thickeners for a reactive dye, in digital printing or inkjet printing the assistants have to be applied to the textile substrate in a separate pretreatment step.

The pretreatment of the textile substrate, such as cellulose fibers and regenerated cellulose fibers, and also silk and wool, for example, takes place prior to printing, using an aqueous alkaline liquor. The fixing of reactive dyes requires alkali, such as sodium carbonate, sodium bicarbonate, sodium acetate, trisodium phosphate, sodium silicate or sodium hydroxide, alkali donors such as, for example, sodium chloroacetate or sodium formate, hydrotropic substances such as, for example, urea, reduction inhibitors, such as, for example, sodium nitrobenzenesulfonates, and also thickeners to prevent the motifs flowing when the printing ink is applied. The latter are, for example, sodium alginates, modified polyacrylates or highly etherified galactomannans.

These pretreatment reagents are applied uniformly to the textile substrate in a defined amount using suitable applicators, examples being a 2- or 3-roll padder, using contactless spraying technologies, by means of foam application, or using appropriately adapted inkjet technologies, and are subsequently dried.

Printing is followed by drying of the textile fiber material at 120 to 150° C. and then by fixing.

The fixing of the inkjet prints prepared with reactive dyes can be carried out at room temperature or with saturated steam, with superheated steam, with hot air, with microwaves, with infrared radiation, with laser or electron beams or with other suitable energy transfer techniques.

A distinction is made between one- and two-phase fixing operations. In one-phase fixing the necessary fixing chemicals are already on the textile substrate. In the case of two-phase fixing this pretreatment is unnecessary. Fixing requires only alkali, which is applied following inkjet printing and before the fixing operation, without drying in between. There is no need for further additions such as urea or thickener.

Fixing is followed by print aftertreatment, which is the prerequisite for good fastnesses, high brilliance and an immaculate white ground.

The prints prepared with the inks of the invention, especially on cellulose fiber materials, possess high color strength and a high fiber-dye bond stability not only in the acidic but also in the alkaline range, and also possess good light fastness and very good wet fastness properties, such as fastness to washing, water, saltwater, cross-dyeing and perspiration, and also good fastness to heat setting and pleating, and crockfastness.

The examples which follow serve to illustrate the invention. Parts and percentages are by weight unless noted otherwise. The relationship of parts by weight to parts by volume is that of the kilogram to the liter. The compounds described by formula in the examples are written in the form of the alkali metal salt, since they are generally prepared and isolated in the form of their salts, preferably sodium or potassium salts, and are used in the form of their salts for coloring. The starting compounds specified in the examples below can be used for synthesis in the form of the free acid or likewise in the form of their salts, preferably alkali metal salts, such as sodium or potassium salts.

### EXAMPLE 1

A suspension of 800 parts of water and 114 parts of the monoazo compound of the formula (4) where M is as defined above, preferably sodium, is adjusted to a pH of 6.5 with sodium carbonate. 36.9 parts of cyanuric chloride are then introduced and the batch is stirred at room temperature for 1 hour while maintaining the pH at 6.0 to 6.5 with 15% sodium carbonate solution. After the acylation has ended, a neutral solution of 102 parts of the monoazo dye of the following formula:

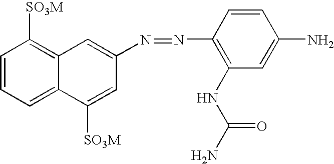

where M is as defined above, in 1000 parts of water is added, the pH is adjusted to 4.0 with concentrated hydrochloric acid and the mixture is subsequently stirred at 70° C. for 4 hours while the pH is maintained at 4.0 by addition of 15% sodium carbonate solution. The invented dye (1-1) is subsequently isolated by evaporating the as-synthesized solution in a vacuum drying cabinet.

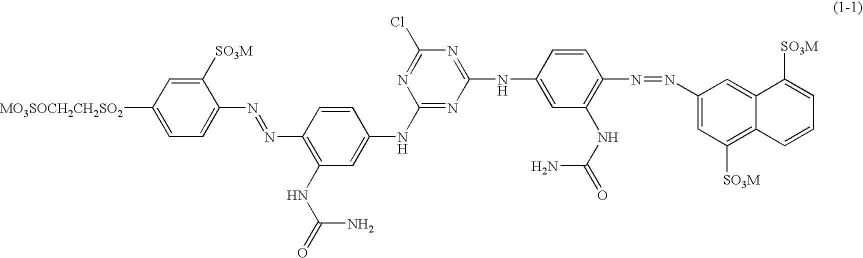

where M is as defined above. When applied and fixed by the methods customary in the art for fiber-reactive dyes, it provides strong reddish yellow dyeing and prints of good light- and wetfastness properties on cotton for example and exhibits a very good buildup performance.

### EXAMPLE 2

114 parts of the monoazo compound of the formula (4) where M is as defined above, preferably sodium, are acylated with cyanuric chloride as described in Example 1. A neutral solution of 122 parts of the monoazo dye of following formula:

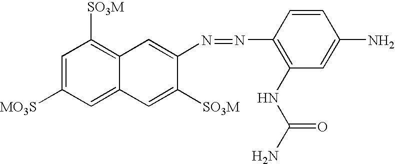

where M is as defined above, in 900 parts of water is subsequently added, the pH is adjusted to 4.0 with concentrated hydrochloric acid and the mixture is subsequently stirred at 70° C. for 5 hours while the pH is maintained at 4.0 by addition of 15% sodium carbonate solution. The invented dye (1-2) is subsequently isolated by evaporating the as-synthesized solution in a vacuum drying cabinet.

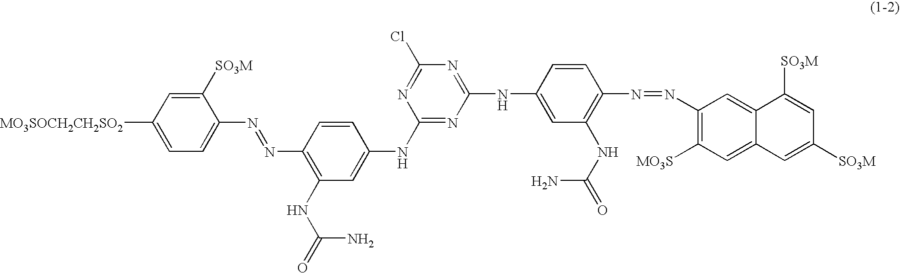

where M is as defined above. When applied and fixed by the methods customary in the art for fiber-reactive dyes, it provides strong reddish yellow dyeing and prints of good light- and wetfastness properties on cotton for example and exhibits a very good buildup performance.

### EXAMPLES 3 TO 8

The table examples hereinbelow describe further reactive dyes conforming to the general formula (1). They are preparable in the manner of the present invention similarly to the operative examples indicated above.

They possess very good dye properties and applied and fixed by the methods customary in the art for fiber-reactive dyes they provide strong dyeing and prints of good light- and wetfastness properties on cotton for example and exhibit a very good buildup performance.

| Example | R | Hue |
|---|---|---|
| 3 | Formula (2c) | reddish yellow |
| 4 | Formula (2d) | ″ |
| 5 | Formula (2e) | ″ |
| 6 | Formula (2f) | ″ |
| 7 | Formula (2g) | ″ |
| 8 | Formula (2h) | ″ |

### EXAMPLE 9

114 parts of the monoazo compound of the formula (4) where M is as defined above, preferably sodium, are acylated with cyanuric chloride as described in Example 1. 48 parts of 2-amino-5-naphthol-7-sulfonic acid are then added, the pH is adjusted to 4.5 with dilute hydrochloric acid and the mixture is subsequently stirred at 45° C. for 4 hours while the pH is maintained at 4.5 by addition of 15% sodium carbonate solution. After the condensation has ended, the mixture is cooled down to room temperature to obtain the compound of the following formula (1-9):

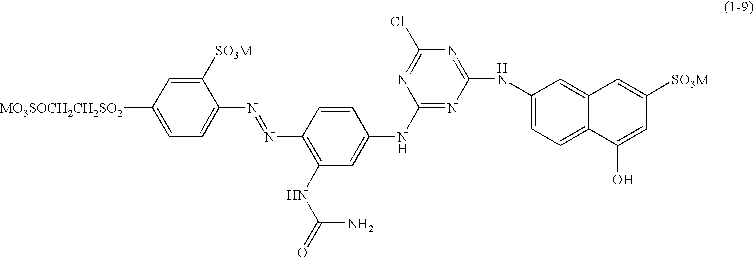

### EXAMPLE 10

50 parts of aniline-2,5-disulfonic acid are suspended in 200 parts of water and dissolved with concentrated sodium hydroxide solution at pH 7, cooled down to 0° C. and admixed with 50 parts of an aqueous 5N sodium nitrite solution. This solution is added dropwise to a mixture of 200 parts of ice and 70 parts of concentrated hydrochloric acid before stirring for 1 hour more. Thereafter, excess nitrite is destroyed with a little sulfamic acid. The dye solution prepared according to Example 9 is added dropwise to the present suspension while the pH is maintained at 6.3 to 6.5 by means of a 15% aqueous sodium carbonate solution. The reaction mixture is subsequently stirred at 18-20° C. for a further 2 hours, then adjusted to pH 5 with a little dilute hydrochloric acid and evaporated under reduced pressure to obtain the dye of the formula (1-10)

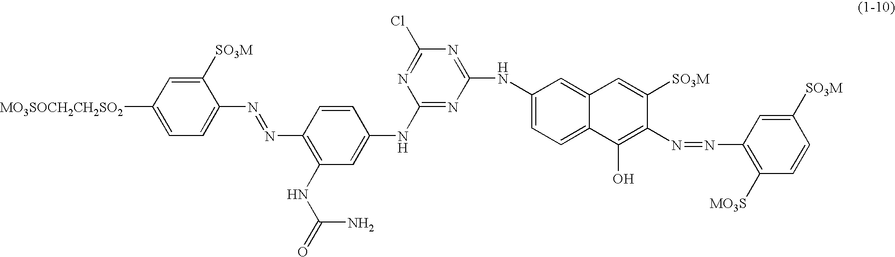

where M is as defined above. Applied and fixed by the methods customary in the art for fiber-reactive dyes it provides strong reddish yellow dyeing and prints of good light- and wetfastness properties on cotton for example and exhibits a very good buildup performance.

### EXAMPLE 11

56 parts of 4-(β-sulfatoethylsulfonyl)aniline are suspended in 200 parts of water and dissolved with sodium bicarbonate at pH 7, cooled down to 0° C. and admixed with 50 parts of an aqueous 5N sodium nitrite solution. This solution is added dropwise to a mixture of 200 parts of ice and 70 parts of concentrated hydrochloric acid before stirring for 1 hour more. Thereafter, excess nitrite is destroyed with a little sulfamic acid. The dye solution prepared according to Example 9 is added dropwise to the present suspension while the pH is maintained at 6.3 to 6.5 by means of a 15% aqueous sodium carbonate solution. The reaction mixture is subsequently stirred at 18-20° C. for a further 2 hours, then adjusted to pH 5 with a little dilute hydrochloric acid and evaporated under reduced pressure to obtain the dye of the formula (1-11)

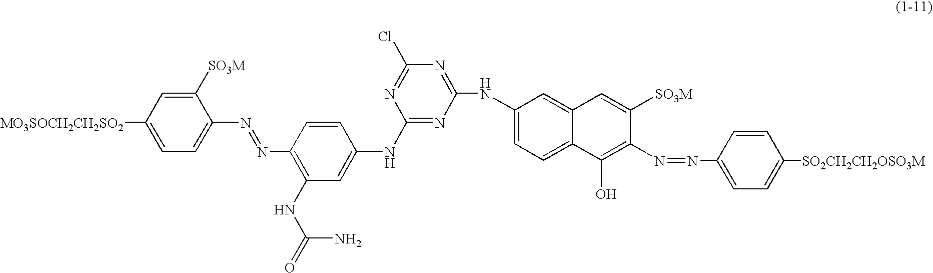

where M is as defined above. Applied and fixed by the methods customary in the art for fiber-reactive dyes it provides strong reddish yellow dyeing and prints of good light- and wetfastness properties on cotton for example and exhibits a very good buildup performance.

### EXAMPLES 12 TO 22

The table examples hereinbelow describe further reactive dyes conforming to the following general formula:

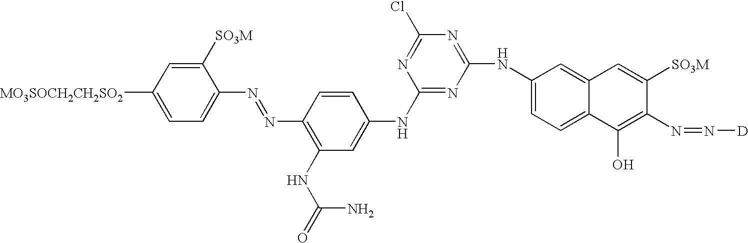

They can be prepared in the manner of the present invention similarly to the above-reported operative examples 9 to 11.

They possess very good dye properties and applied and fixed by the methods customary in the art for fiber-reactive dyes they provide strong dyeing and prints of good light- and wetfastness properties on cotton for example and exhibit a very good buildup performance.

| Example | D | Hue |
|---|---|---|
| 12 | 2,4-disulfophenyl | reddish yellow |
| 13 | 2,5-disulfo-4-methylphenyl | ″ |
| 14 | 2-sulfophenyl | ″ |
| 15 | 3-sulfophenyl | ″ |
| 16 | 4-sulfophenyl | ″ |
| 17 | 4,8-disulfo-2-naphthyl | orange |
| 18 | 1,5-disulfo-2-naphthyl | ″ |
| 19 | 6,8-disulfo-2-naphthyl | ″ |
| 20 | 3,6,8-trisulfo-2-naphthyl | ″ |
| 21 | 3-(β-sulfatoethylsulfonyl)phenyl | yellowish orange |
| 22 | 2-chloro-4-(β-sulfatoethylsulfonyl)- | reddish yellow |
| | phenyl | |

### EXAMPLE 23

A textile fabric consisting of mercerized cotton is padded with a liquor containing 35 g/l of calcined sodium carbonate, 100 g/l of urea and 150 g/l of a low-viscosity sodium alginate solution (6%) and then dried. The liquor pickup is 70%. The textile thus pretreated is printed with a pattern using an aqueous ink containing 2% of the dye mixture according to Example 1, 20% of sulfolane, 0.01% of Mergal K9N and 77.99% of water, and using a drop-on-demand (bubblejet) inkjet printing head. The print is fully dried. Fixing takes place by means of saturated steam at 102° C. for 8 minutes. Thereafter the print is rinsed warm, subjected to a fastness wash with hot water at 95° C., rinsed warm and then dried. This gives a yellow print having an outstanding durability.

### EXAMPLE 24

A textile fabric consisting of mercerized cotton is padded with a liquor containing 35 g/l of calcined sodium carbonate, 50 g/l of urea and 150 g/l of a low-viscosity sodium alginate solution (6%) and then dried. The liquor pickup is 70%. The textile thus pretreated is printed with a pattern using an aqueous ink containing 8% of the dye mixture according to Example 2, 20% of 1,2-propanediol, 0.01% of Mergal K9N and 71.99% of water, and using a drop-on-demand (bubblejet) inkjet printing head. The print is fully dried. Fixing takes place by means of saturated steam at 102° C. for 8 minutes. Thereafter the print is rinsed warm, subjected to a fastness wash with hot water at 95° C., rinsed warm and then dried. This gives a yellow print having an outstanding durability.

### EXAMPLE 25

A textile fabric consisting of mercerized cotton is padded with a liquor containing 35 g/l of calcined sodium carbonate, 100 g/l of urea and 150 g/l of a low-viscosity sodium alginate solution (6%) and then dried. The liquor pickup is 70%. The textile thus pretreated is printed with a pattern using an aqueous ink containing 8% of the dye mixture according to Example 3, 15% of N-methylpyrrolidone, 0.01% of Mergal K9N and 76.99% of water, and using a drop-on-demand (bubblejet) inkjet printing head. The print is fully dried. Fixing takes place by means of saturated steam at 102° C. for 8 minutes. Thereafter the print is rinsed warm, subjected to a fastness wash with hot water at 95° C., rinsed warm and then dried. This gives a yellow print having an outstanding durability.

1-6. (canceled)

7. A dye of the formula (1) 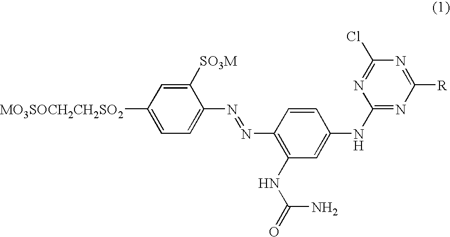
where
M is hydrogen or an alkali metal,
R is a monoazo dye of the formulae (2a) to (2i) 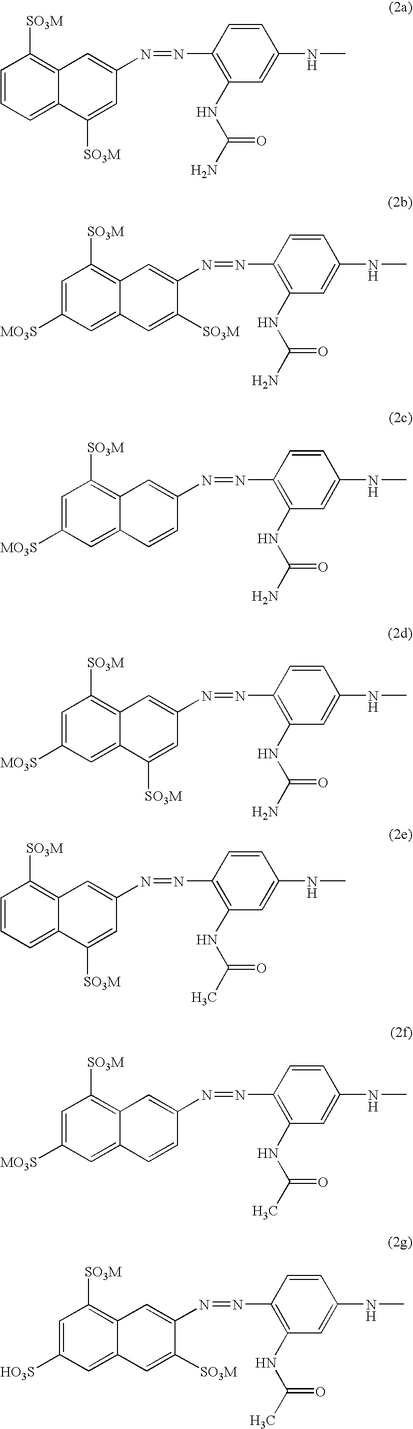 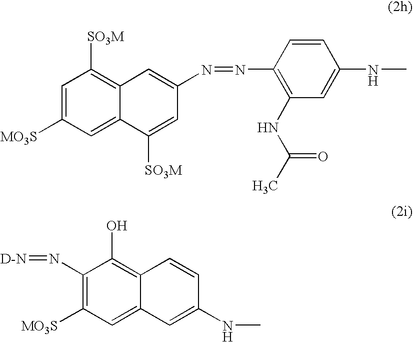
wherein
D is a group of the general formula (3) 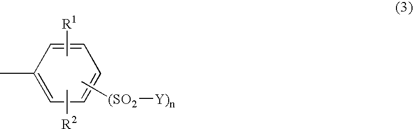
where
R¹ is hydrogen, methyl, methoxy, sulfo or chlorine,
R² is hydrogen, methyl, methoxy, sulfo or chlorine,
n is zero or 1 (this group being hydrogen in the case of n being zero) and
Y is vinyl or is ethyl substituted in the P position by a substituent which is alkali eliminable to leave a vinyl group.

8. The dye as claimed in claim 7, wherein
M is hydrogen sodium, potassium or lithium and
Y is chlorine, bromine, acetyloxy, p-tolylsulfonyloxy, thiosulfato, phosphate, sulfato or β-hydroxyethyl.

9. The dye as claimed in claim 8, wherein
Y is sulfato or β-hydroxyethyl.

10. A process for preparing a dye of the general formula (1) 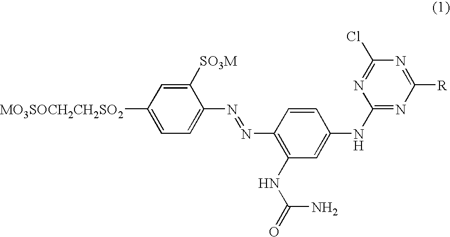
wherein
M is hydrogen or an alkali metal,
R is a monoazo dye of the formulae (2a) to (2i) 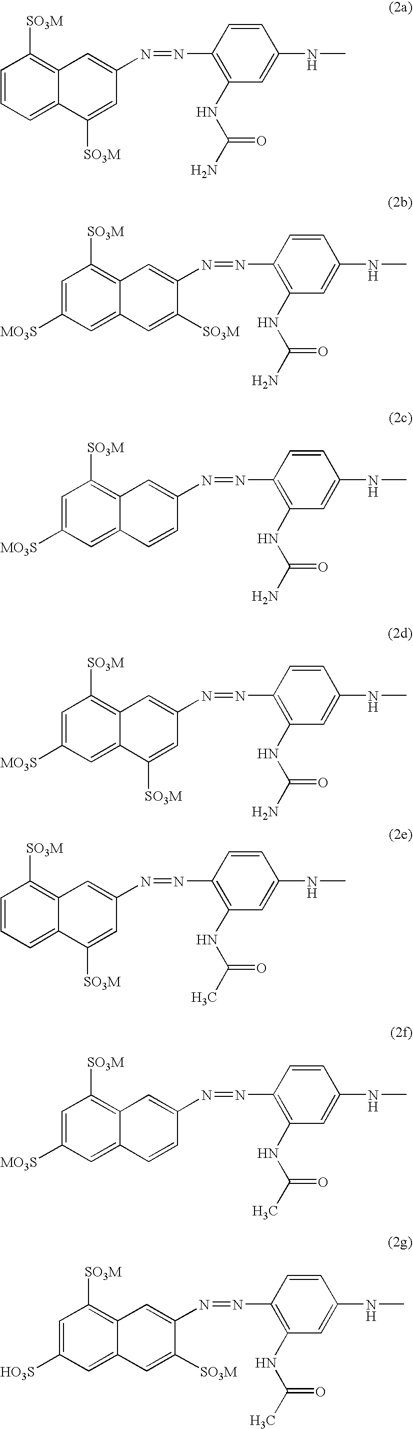 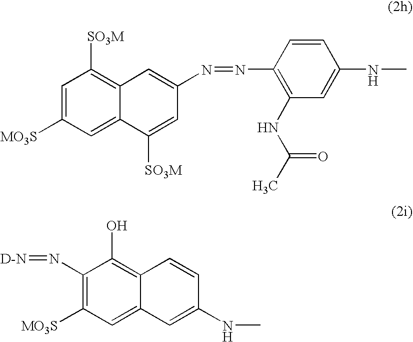
wherein
D is a group of the general formula (3) 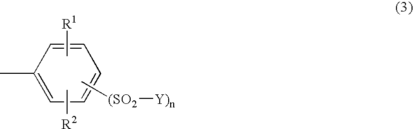
wherein
R¹ is hydrogen, methyl, methoxy, sulfo or chlorine,
R² is hydrogen, methyl, methoxy, sulfo or chlorine,
n is zero or 1 (this group being hydrogen in the case of n being zero) and
Y is vinyl or is ethyl substituted in the P position by a substituent which is alkali eliminable to leave a vinyl group,
which comprises acylating the monoazo compound of the formula (4) 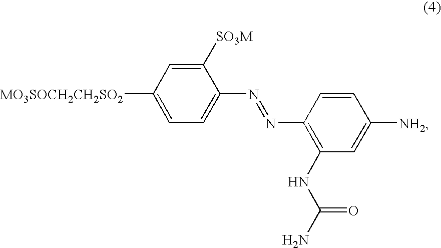
where M is as defined above, with cyanuric chloride and subsequently condensing with a monoazo dye of the formula (5) or formula (6)
R—H (5) 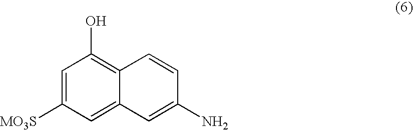
where R and M are as defined above and if the monoazo dye of the formula (6) is used then the resulting condensation product is subsequently reacted with the diazonium compound of an amine of the general formula (7)
D-NH₂ (7)
where D is as defined above.

11. The process as claimed in claim 10, wherein
R is a monoazo dye of the formula (2i)
said condensing with the compound of the formula (6) and the resulting condensation product subsequently being reacted with the diazonium compound of the amine of the general formula (7).

12. The process as claimed in claim 10, wherein
R is a monoazo dye of the formulae (2a) to (2h) and said condensing is with the monoazo dye of the formula (5).

13. A process to print or dye a hydroxyl- and/or carboxamido-containing material which comprises printing or dyeing the material with the dye according to claim 7.

14. An aqueous digital printing ink comprising the dye mixture according to claim 7.

15. Hydroxyl- and/or carbonamido-containing material which comprises the hydroxyl- and/or carbonamido-containing material dyed or printed with the dye according to claim 7.
